(19) United States
(12) Patent Application Publication
(10) Pub. No.: US 2004/0068193 A1
(43) Pub. Date: Apr. 8, 2004

(54) Optical devices for medical diagnostics

(76) Inventors: Russell H. Barnes, Columbus, OH (US); Busolo W. Wabuyele, Columbus, OH (US)

Correspondence Address: Battelle Memorial Institute, 505 King Avenue, Columbus, OH, 43201-2693, US

(21) Appl. No.: 10/632,387

(22) Filed: Aug. 1, 2003

Related U.S. Application Data
(60) Provisional application No. 60/400,791, filed on Aug. 2, 2002.

(51) Int. Cl. A61B 6/00

(52) U.S. Cl. 600/476

(57) ABSTRACT

The invention discloses apparatus and methods that combine fluorescence spectra, Raman spectra, and diffuse reflectance, with image analysis in a single instrument package along with algorithms and data processing software. The data processing software involves spectral analysis, image analysis, data compression, data fusion, and classification processes. A spectral database with optical tissue characteristics is developed from a pathology laboratory and/or from in vivo measurements to provide the information required to differentiate normal and abnormal tissue.

This application claims the benefit of US Provisional Application Serial No. 60/400,791 filed Aug. 2, 2002, to Barnes and Wabuyele.

The entire disclosure of the provisional application is incorporated herein by reference as if completely rewritten herein.

### FIELD OF THE INVENTION

The present invention provides for an apparatus and method for substantially simultaneous measurement of fluorescence, Raman spectra, and diffuse reflectance (transflectance) of a target tissue, and image analysis combined in a single instrument. The invention allows rapid diagnostic measurements having high reliability.

### BACKGROUND OF THE INVENTION

As a result of laser fluorescence studies in 1987 on arterial samples from human cadavers supplied by The Ohio State University, and other tissues studies since that time, it is apparent that optical spectroscopy has the potential for rapid biopsy of tissues plus providing more extensive histological information than is normally available from routine pathology laboratory results. Since the early work in 1987, the term “optical biopsy” has come into common use representing an active area of research today. One obvious area of application is the detection and diagnosis of cancer. Optical methods can be readily adapted to surgical procedures to detect, classify, and verify complete removal of cancerous tissue.

Recent investigations have demonstrated the utility of optical spectroscopy for the detection of abnormalities in human tissue. Techniques based on Raman, fluorescence, diffuse-reflectance spectroscopy and other techniques have been shown to provide valuable information on tissue pathology. An opportunity exists to integrate multispectral techniques with image processing to develop an evolutional line of clinical instruments for optical biopsy.

### BRIEF DESCRIPTION OF THE INVENTION

The invention broadly includes a diagnostic device and method combining Raman, fluorescence and transflectance measurements for the detection and identification of cancerous tissues and tumors.

A broad embodiment provides for Raman, fluorescence, and transflectance measurements covering substantially the same region such as surface area, or surface volume (typically e.g. in the case of depth profiling) to improve disease (e.g. cancer) detection and diagnostics, to improve the accuracy for identification of normal, precancerous, and types of cancerous tissues; and benign and cancerous tumors. Typically fusion of the Raman, fluorescence, and transflectance measurements is performed.

A first embodiment of the invention provides for a method for diagnosing disease in a patient by the steps of (a) generating data for Raman, fluorescence, and diffuse reflectance spectra and images for a region of a selected tissue of the patient; (b) providing data as a spectral library of tissue database classified by normal and diseased tissue for Raman, fluorescence and diffuse reflectance spectra and images for the same type of tissue from other individuals, typically in substantially the same region; and (c) performing classification decisions to detect and identify diseased tissue by comparing the generated data from step a with the provided data in step (b). Another embodiment includes an additional step of (d) displaying image and data results to a user as to the identification of the state of disease for the patient's selected tissue. Typically the generated data of step (a) is obtained by biopsy or direct optical measurement of the patient's tissue and the data for the spectral library is developed from biopsy (ex vivo) or direct measurement (in vivo) from other individuals, again typically in the substantially the same region of the tissue. Yet other embodiments provide for (1) fusing the generated data for Raman, fluorescence, and diffuse reflectance of step a; and (2) fusing the provided data for Raman, fluorescence, and diffuse reflectance of step b, typically the fusing steps are performed prior to performing the classification decisions of step c.

The patient is typically a mammal, and in some embodiments a human being; horse, dog, or other domesticated animal.

A further embodiment of the invention includes a method for diagnosing disease in a patient by the steps of (a) generating illumination light for Raman, fluorescence, and diffuse reflectance measurements (light generation may be sequential); (b) illuminating a region of selected tissue of a patient with the light generated in step (a); (c) generating data for Raman, fluorescence, and diffuse reflectance spectra and images for a region of the selected tissue of the patient; (d) providing data as a spectral library of tissue database classified by normal and diseased tissue for Raman, fluorescence and diffuse reflectance spectra and images for the same type of tissue from other individuals (typically from substantially the same region of the tissue); and (e) performing classification decisions to detect and identify diseased tissue by comparing the generated data from step c with the provided data in step (d). A further embodiment includes the additional step (f) displaying image and data results to a user as to the identification of the state of disease for the patient's selected tissue. Typically the generated data of step (c) is obtained by biopsy or direct optical measurement of the patient's tissue and the data for the spectral library is developed from biopsy (ex vivo) or direct measurement (in vivo) from other individuals. Yet another embodiment of the invention provides for (1) fusing the generated data for Raman, fluorescence, and diffuse reflectance of step c; and (2) fusing the provided data for Raman, fluorescence, and diffuse reflectance of step d; wherein the fusing steps are performed prior to performing the classification decisions of step e.

Another broad embodiment of the invention includes apparatus for identifying and detecting the disease state of a patient's selected tissue including (a) means for generating light adapted to generate Raman, fluorescence, and diffuse reflectance spectra and images for a region of the selected tissue of the patient (light generation may be sequential); (b) means for illuminating a region of a patient's tissue with the generated light; (c) means for collecting light emanating from the illuminated tissue; (d) means for providing Raman, fluorescence and diffuse reflectance spectra and images from the collected light; (e) a spectral library of Raman, fluorescence, and diffuse reflectance spectra and images representative of normal and diseased tissue (typically for substantially the same region of the tissue); and (f) a computer system for controlling light generation in a above, and detecting and classification of the patient's tissue based on information from the means for detecting of d, e, and f above, and the spectral library. Additional embodiments include means for displaying data and/or images from the computer system, perceptible to a user, as to the identification of the state of disease for the patient's selected tissue. Yet additional embodiments include (1) means for fusing the Raman, fluorescence, and diffuse reflectance spectra and images of means d; and (2) means for fusing the spectral library Raman, fluorescence, and diffuse reflectance spectra and images; wherein the means for fusing are prior to the computer system performing the classification decisions.

Typically the invention provides for the use of time gating to reduce interferences from surface scattering and to reduce and/or remove interferences between Raman and fluorescence measurements. Typically the use of time gating also allows for depth profiling below the surface of tissues and tumors.

### BRIEF DESCRIPTION OF THE DRAWINGS

FIG. 1 is of a schematic drawing of one embodiment of the invention for apparatus showing the various parts of the light detection system and their interrelationship.

FIG. 2 is a schematic drawing of apparatus for analyzing the spectral data obtained from the apparatus according to FIG. 1.

FIG. 3 shows typical time gating for Raman and fluorescence spectra. The vertical scale is intensity (I) and the horizontal scale is time (t).

FIG. 4 is a schematic drawing showing typical paths for diffuse reflected light within tissue.

FIG. 5 is a typical curve for Raman spectra as a function of intensity (vertical scale) and Raman shift, cm⁻¹, (horizontal scale).

FIG. 6 shows a typical diffuse reflectance spectrum plotted as a function of absorbance, A,(vertical scale) and wavelength, λ, (horizontal scale).

FIG. 7 is a typical excitation emission spectra matrix (EEM) that is a three dimensional surface made up of multiple curves of fluorescence spectra. The vertical scale is in intensity (I), the left scale is excitation wavelength (λ_{exc}) and the right scale is emission wavelength (λₑₘₘᵢ).

### DETAILED DESCRIPTION OF THE INVENTION AND BEST MODE

Broadly the present invention provides for a diagnostic device including means for Raman, fluorescence and transflectance measurements, means for data computation, and means for display typically perceptible to a user for the detection and identification of disease. The device typically combines the three techniques, fluorescence, Raman, and diffuse reflectance, with spectral and image analysis in a single instrument package with the necessary algorithms and data processing software. The data processing software typically involves spectral analysis, image analysis, data compression, data fusion, and classification processes. A spectral library database with optical tissue characteristics is developed from a pathology laboratory and/or from in vivo measurements to provide the information required to differentiate normal and abnormal tissue.

The apparatus and method have wide applications in that a variety of tissues can be diagnosed for various diseases as long as optical access to the tissue is available. Typically cancers such as digestive tract cancers (e.g. esophageal, stomach and bowel cancer), cervical cancer, skin cancer, bladder cancer, breast cancer, and prostate cancer are diagnosable with the invention.

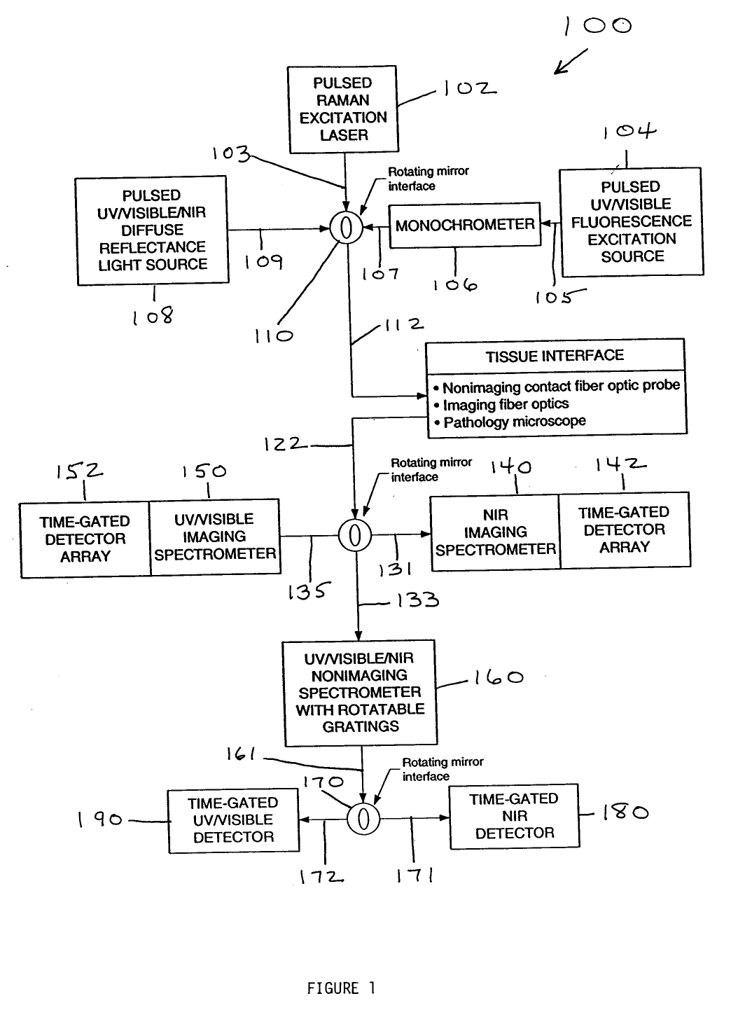

Referring now to FIG. 1, this figure illustrates one embodiment of the invention that includes integrated optical biopsy apparatus 100. This first embodiment of the invention includes three sources of light. A first light source 102 provides light for measuring Raman spectra and is typically a pulsed Raman excitation laser. The pulsed Raman laser includes pulsed solid state or gas lasers used to excite Raman spectra from tissue. Wavelengths of interest range from 550 nm to 2.5 μm to minimize fluorescence interference. A pulse laser is used for time-gated detection to optimize minimization of fluorescence interference, reduction of tissue surface scattering and for depth profiling in tissue. The pulsed Raman excitation laser is preferably operated at a wavelength of about 700 nm to about 1100 nm. More preferably the pulsed Raman excitation laser is operated at a wavelength of about 700 nm to about 800 nm.

A second source of light 104 provides light for measuring for fluorescence measurements of tissue. It is typically a pulsed ultraviolet (UV) or visible light source that excites fluorescence in the tissue under observation. More particularly, the second light source 104 is typically a broadband light source or tunable light source for excitation of fluorescence from tissues in the ultraviolet, visible and near-infrared regions (NIR) of the spectrum. Broadband light sources would include incandescent and discharge sources such as rare gas lamps including xenon, deuterium, krypton, and mercury lamps. Tunable sources would include tunable dye lasers, Raman lasers, optical parametric oscillators, and nonlinear frequency doubling techniques. The broadband light output 105 of second light source 104 typically is sent to an optional monochrometer 106.

Optional monochrometor 106 is for use with broadband light sources. It is intended to scan or select wavelengths for excitation of fluorescence and provide excitation wavelength scans for recording synchronous fluorescence spectra and excitation-emission matrix (EEM) three-dimensional fluorescence spectra.

A third source of light 108 is a pulse diffuse reflectance light source This light source would include modulated broadband incandescent and pulsed discharge sources for diffuse reflectance or transflectance measurements with tissues. The pulsed source is to allow time gating to reduce surface scattering effects and allow depth profiling.

First rotating mirror interface is a tilted rotating mirror assembly that allows the three light sources; 102, 104 and 108 to be interfaced individually and typically sequentially with a fiber optic or light guide to the tissue interface. Light from the rotating mirror interface 110 is sent via illuminating fiber optic cable 112 to the tissue interface 120. Light beams 103, 107, 109 go to the first rotating mirror interface 110 from the three light sources 102, 104, 108 respectively or beam 107 may come from the optional monochrometer 106. The light beams may travel in typical optical fiber assemblies and optical transfer systems.

Illuminating optical fiber assembly or light guide 112 conveys light from the rotating mirror interface 110 to the tissue interface 120 for tissue illumination. The fiber optic assembly is to be compatible with the configuration and use of the tissue interface 120. Fiber optic cables typically used in medical instrument industry are useful in the invention.

The tissue interface 120 may be in the form of (1) a nonimaging contact fiber optic probe, (2) imaging fiber optic probe, (3) endoscope, and/or (4) imaging interface for a pathology microscope.

Light returning from the tissue under observation impinges the tissue inter interface 120 and is sent via return fiber optic cable 122 to a second rotating mirror interface 130. This second rotating mirror interface 130 selectively or sequentially transfers light and/or images to the three spectral analysis and image analysis modules (140/142, 150/152, 160) in the integrated optical biopsy system 100.

Light beams 131, 133, 135, containing imaging and nonimaging tissue information from the second rotating mirror interface 130, go to the three spectral/image analysis modules 140, 150 and 160. The light beams may travel in typical optical fiber assemblies and optical transfer systems.

Near infrared (NIR) imaging spectrometer 140 and time-gated detector array 142, in combination, typically record time-gated spectral images. Outputs typically include NIR spectra for diffuse reflectance associated with individual pixels or images over a spectral bandwidth typically centered at a selected wavelength.

Ultraviolet (UV)/Visible imaging spectrometer 150 with time-gated detector array 152 in combination typically provide time-gated spectral images. Outputs typically include Raman and fluorescent spectra for individual pixels or images over a spectral bandwidth typically centered at a selected wavelength.

Nonimaging spectrometer 160 is a spectrometer with a rotating turret with selectable gratings optimized for UV, visible and NIR regions of the spectrum. Total collected radiation from the fluorescence, Raman, and diffuse reflectance is analyzed in a nonimaging mode to supply high sensitivity spectra.

Light 161 from the non imaging spectrometer with rotable gratings 160 is sent to a third rotating mirror interface 170 that splits the light into two beams 171, 172. This interface is typically a fiber optical or optical transfer system that outputs from the nonimaging spectrometer with rotable gratings 160 to either a time-gated UV-visible detector 190 or a time-gated NIR detector 180. The first beam 171 goes to time gated near infrared detector 180 that typically detects diffuse reflectance. The second light beam 172 goes to a time gated ultraviolet/visible detector 190.

The time-gated NIR detector 180 typically is a single element NIR detector with response over the spectral region from 0.9 to 2.5 μm. Time gating typically serves to reduce surface reflectance and/or provide for tissue depth profiling.

The Time-Gated UV/Visible Detector 190 is typically a high sensitivity single element detector such as a photomultiplier covering the spectral range from 200 to 900 nm for fluorescence, Raman and diffuse reflectance measurements. Time gating is typically used to reduce surface reflectance for tissue depth profiling and/or for fluorescence lifetime discrimination.

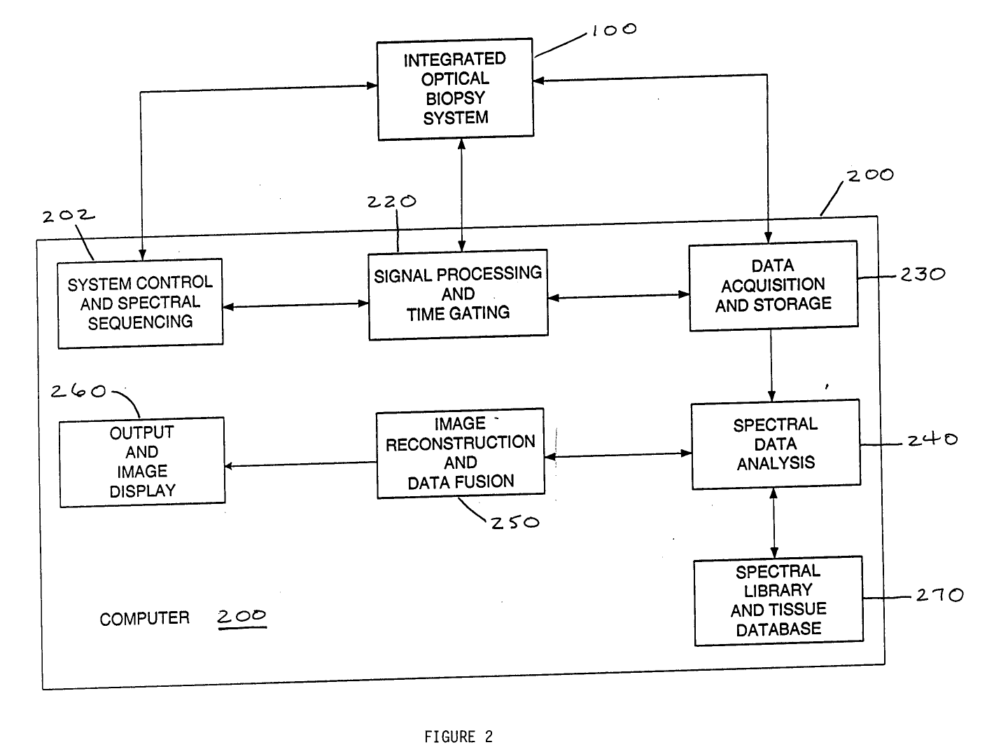

Referring now to FIG. 2, this figure illustrates a schematic diagram of a typical computer system 200 for control of the integrated optical biopsy apparatus 100 and data processing.

The System Control and Spectral Sequencing module 202 typically consists of software written for the system that provides overall control for the integrated optical biopsy apparatus 100, preparing the apparatus 100 for operation and invoking each of the spectral collection methods in turn. This high-level application invokes low-level routines that provide control of the system hardware, such as the stepper motor components and controls the rotating mirrors 110, 130 and 170 that coordinate selection of the light source and spectral/image analysis module combinations. The system control application interfaces routes output signal to 220 for signal processing.

The signal processing and time gating module 220 receives input from 202 that is processed and time gated and transferred to data acquisition and storage module 230 where data are acquired and stored. Typical Signal processing includes averaging, filtering and application of noise reduction and signal enhancement techniques. Signal processing techniques, such as time gating, will be utilized during the data collection stages for data enhancement, reduction of surface scattering, and tissue depth profiling.

The data acquisition and storage module 230 typically accepts information from the apparatus 100 and signal processing and time gating module 220. A bifurcated optical fiber bundle 112, 122 will interface the apparatus integrated spectroscopic system with a tissue sample. Stepped motors (not shown) controlled by computer 200 will be used to change optical elements so that fluorescence, diffuse reflectance, and Raman spectra can be acquired, processed and recorded automatically without moving the sample so that the spectra from the three optical modalities are recorded on substantially the same region. Multiple spectra are typically recorded on multiple normal and abnormal regions of each tissue sample to accumulate appropriate statistically information for design of algorithms and analysis of reliability probabilities.

The spectral data analysis module 240 typically provides for specialized spectral analysis techniques using multivariate pattern recognition/classification and discriminating algorithms for automatic detection and classification of benign, pre-cancerous, and cancerous tissue with high sensitivity and specificity. The spectral information will be correlated in real-time to tissue pathology in module 270 to improve the classification accuracy and reliability. The effect of spectral outliers and pre-treatments on tissue classification will be evaluated. These will include but not limited to spectral normalization techniques, mean- centering, auto-scaling, variance scaling and range scaling. The spectral data analysis output from module 240 will consist of classification results including sensitivity and specificity values obtained from the individual Raman, fluorescence, and diffuse reflectance techniques.

The spectral library and tissue database 270 typically provides spectra and images of samples (e.g. ex-vivo samples) that are recorded on a full range of normal, pre-cancerous, and cancerous types of tissue and tumors to provide a spectral library and tissue database for the identification and classification of a selected range of cancerous tissue. Tissue samples will be accompanied by pathology information, identification and microscopic photographic images. The spectral analysis module 240 with training or calibration algorithms will use this information for validation, improvement and optimization of the final classification algorithms in module 240.

The image reconstruction and data fusion module 250 typically uses data compression/fusion and image reconstruction techniques, spectral analysis information from three optical techniques, and image analysis combined into a single software module. The input from module 240 is fused with the information from Raman, fluorescent, and diffuse reflectance measurements and the image analysis results. This enables automated, fast, and accurate pre-cancer screening and diagnosis.

In the output and image display 260, results from classification algorithms from image reconstruction and data fusion module 250 will be presented. This will typically include the use of standard figures and merit tables (e.g. error rates, confusion matrices, sensitivity and specificity) to display and summarize classification results.

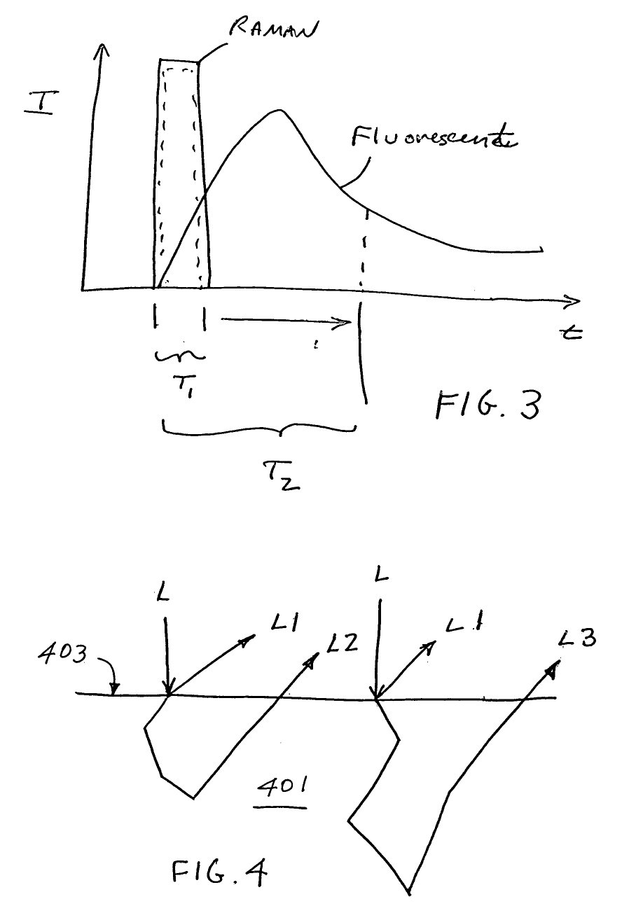

FIG. 3 shows typical time gating for Raman and fluorescence spectra. The vertical scale is intensity (I) and the horizontal scale is time (t). Typical time gates for Raman spectra and fluorescence spectra are shown as T₁ and T₂ respectively.

FIG. 4 is a schematic drawing showing typical paths for reflected light within tissue 401. Time gating is useful for eliminating surface reflected light (L1) at tissue surface 403, from reflected light that enters tissue (L2 and L3) to obtain better information provided by penetration of light to various depths within the tissue 401. L2 and L3 are typical paths for diffused reflected light at different depths.

FIG. 5 is a typical curve for Raman spectra as a function of intensity and Raman shift, cm⁻¹. These spectra are used in modules 240 and 250 for training and classification algorithms.

FIG. 6 shows a typical diffuse reflectance spectrum plotted as a function of absorbance and wavelength. These spectra are used in modules 240 and 250 for training and classification algorithms.

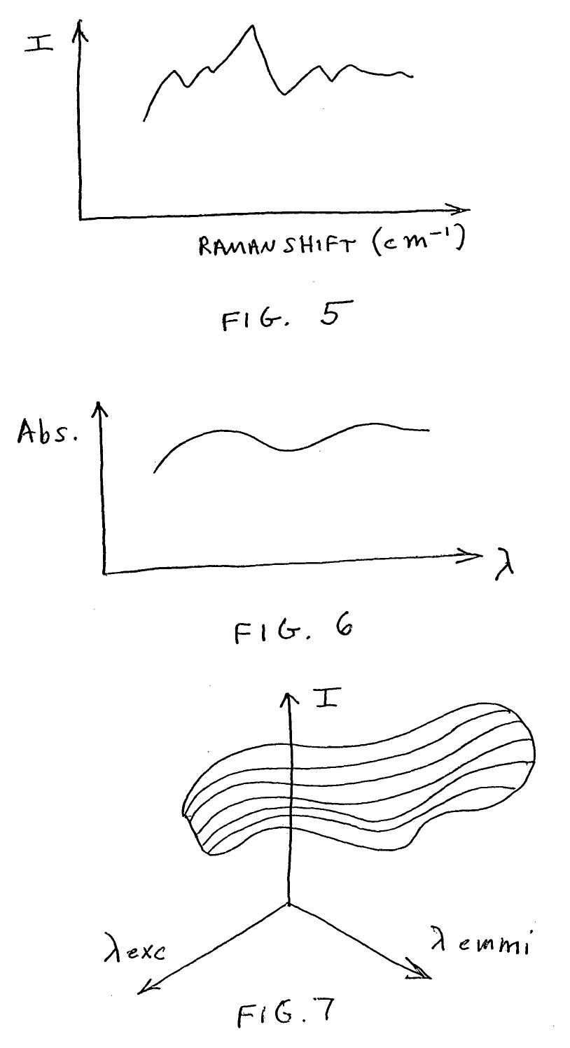

FIG. 7 is a typical excitation emission spectra matrix (EEM) that is a three dimensional surface made up of multiple curves of fluorescence spectra. The EEMs are used in modules 240 and 250 for training and classification algorithms.

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the scope of the invention.

We claim:

1. A method for diagnosing disease in a patient comprising:
a. generating data for Raman, fluorescence, and diffuse reflectance spectra and images for a selected tissue of the patient;
b. providing data as a spectral library of tissue database classified by normal and diseased tissue for Raman, fluorescence and diffuse reflectance spectra and images for the same type of tissue; and
c. performing classification decisions to detect and identify diseased tissue by comparing the generated data from step a with the provided data in step b.

2. The method according to claim 1, with the additional step of:
d. displaying image and/or data results perceptible to a user as to the identification of the state of disease for the patient's selected tissue.

3. The method according to claim 1, wherein the generated data of step a is obtained by biopsy or direct optical measurement of the patient's tissue.

4. The method according to claim 1, wherein the data for the spectral library is developed from biopsy (ex vivo) or direct measurement (in vivo).

5. The method according to claim 1, comprising
(1). fusing the generated data for Raman, fluorescence, and diffuse reflectance of step a; and
(2). fusing the provided data for Raman, fluorescence, and diffuse reflectance of step b;
wherein the fusing steps are performed prior to performing the classification decisions of step c.

6. The method according to claim 1, wherein time gating is used to reduce interferences from surface scattering and/or to reduce or remove interferences between Raman and fluorescence measurements.

7. The method according to claim 1, wherein time gating is used for depth profiling below the surface of tissue.

8. A method for diagnosing disease in a patient comprising:
a. generating illumination light for Raman, fluorescence, and diffuse reflectance measurements;
b. illuminating a region of selected tissue of a patient with the light generated in step a;
c. generating data for Raman, fluorescence, and diffuse reflectance spectra and images from the illumination of selected tissue of the patient;
d. providing data as a spectral library of tissue database classified by normal and diseased tissue for Raman, fluorescence and diffuse reflectance spectra and images for the same type of tissue; and
e. performing classification decisions to detect and identify diseased tissue by comparing the generated data from step c with the provided data in step d.

9. The method according to claim 8, with the additional step of:
f. displaying image and/or data results perceptible to a user as to the identification of the state of disease for the patient's selected tissue.

10. The method according to claim 8, wherein the generated data of step a is obtained by biopsy or direct optical measurement of the patient's tissue.

11. The method according to claim 8, wherein the data for the spectral library is developed from biopsy (ex vivo) or direct measurement (in vivo).

12. The method according to claim 8, comprising
(1). fusing the generated data for Raman, fluorescence, and diffuse reflectance of step c; and
(2). fusing the provided data for Raman, fluorescence, and diffuse reflectance of step d; wherein the fusing steps are performed prior to performing the classification decisions of step e.

13. The method according to claim 8, wherein time gating is used to reduce interferences from surface scattering and to reduce or remove interferences between Raman and fluorescence measurements.

14. The method according to claim 8, wherein time gating is used for depth profiling below the surface of tissue.

15. Apparatus for identifying and detecting the disease state of a patient's selected tissue comprising:
a. means for generating light adapted to generate Raman, fluorescence, and diffuse reflectance spectra and images for a selected tissue of the patient;
b. means for illuminating a region of a patient's tissue with the generated light;
c. means for collecting light emanating from the illuminated tissue;
d. means for providing Raman, fluorescence, and diffuse reflectance spectra and images in the collected light;
e. a spectral library of Raman, fluorescence, and diffuse reflectance spectra and images representative of normal and diseased tissue; and
f. a computer system for controlling light generation in a above, and detection and classification of the patient's tissue based on information from the means for detecting, and the spectral library.

16. Apparatus according to claim 15, comprising:
(g). means for displaying data and/or images from the computer system, perceptible to a user as to the identification of the state of disease for the patient's selected tissue.

17. The apparatus according to claim 15, comprising
(1). means for fusing the Raman, fluorescence, and diffuse reflectance spectra and images of means d; and
(2). means for fusing the spectral library Raman, fluorescence, and diffuse reflectance spectra and images; wherein the means for fusing are prior to the computer system performing the classification decisions.

18. The apparatus according to claim 15, comprising means for time gating data to reduce interferences from surface scattering and/or to reduce or remove interferences between Raman and fluorescence measurements.

19. The apparatus according to claim 15, means for time gating for depth profiling below the surface of tissues.

20. Diagnostic device comprising: apparatus including means for Raman, fluorescence and transflectance measurements, means for data computation, and means for display perceptible to a user for the detection and identification of disease.
